(19) United States
(12) Patent Application Publication
(10) Pub. No.: US 2007/0128667 A1
(43) Pub. Date: Jun. 7, 2007

(54) Secreted neural apoptosis inhibiting proteins

(75) Inventors: Jean Merrill, Whippany, NJ (US); Zhengbin Yao, Sugar Land, TX (US); Wayne Petko, South Bound Brook, NJ (US); Olga Khorkova, North Haledon, NJ (US); George Keesler, Hillsborough, NJ (US); Min Wang, Blue Bell, PA (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 10/596,221

(22) PCT Filed: Nov. 18, 2004
(86) PCT No.: PCT/US04/38671

§ 371 Date: Jun. 5, 2006

Related U.S. Application Data
(60) Provisional application No. 60/529,575, filed on Dec. 16, 2003.

(51) Int. Cl. G01N 33/567; A61K 38/55; A61K 31/727

(52) U.S. Cl. 435/7.2; 514/12; 514/56

(57) ABSTRACT

A novel neuroprotectant was identified by microarray analysis that is differentially expressed between the ventricular zone and the cortex of human adult and fetal brain. The secreted protein antagonizes Wnt action in Xenopus embryos. Methods are described for modulating free radical neurotoxicity by contacting cells with the protein, treating neuronal diseases associated with free radical-mediated cell death by administering the protein, determining neuroprotective genomic targets associated with select free radical toxicity pathways by screening with the protein and using the protein to identify other compounds that modulate the biological activity of the secreted protein and the cell machinery that reacts to the secreted protein.

### FIELD OF INVENTION

This invention is in the field of molecular biology and in particular relates to the identification of a novel neuroprotectant that is capable of modulating the effects of free radical-mediated cell death.

### BACKGROUND OF THE INVENTION

Wnt and Frizzled

Extracellular signaling molecules have essential roles as inducers of cellular proliferation, migration, differentiation and tissue morphogenesis during normal development (Finch et al., Proc. Natl. Acad. Sci. USA (1997) 94:6770-75). In addition, such molecules function as regulators of apoptosis, the programmed cell death that plays a significant role in normal development and functioning of multicellular organisms. When disregulated, signaling molecules and apoptosis are involved in the pathogenesis of numerous diseases, see e.g., Thompson, Science (1995) 267:1456-1462.

Apoptosis is involved in a variety of normal and pathogenic biological events and can be induced by a number of unrelated stimuli. Recent studies of apoptosis have implied that a common metabolic pathway leading to cell death may be initiated by a wide variety of signals, including hormones, serum growth factor deprivation, chemotherapeutic agents, ionizing radiation and infection by human immunodeficiency virus (HIV), (Wyllie, Nature (1980) 284:555-556; Kanter et al., Biochem. Biophys. Res. Commun. (1984) 118:392-399; Duke & Cohen, Lymphokine Res. (1986) 5:289-299; Tomei et al., Biochem. Biophys. Res. Commun. (1988) 155:324-331; Kruman et al., J. Cell. Physiol. (1991) 148:267-273; Ameisen & Capron, Immunol. Today (1991) 12:102-105; and Sheppard & Ascher, J. AIDS (1992) 5:143-147). Agents that affect the biological control of apoptosis thus have therapeutic utility in numerous clinical indications.

While many genes and gene families that participate in different stages of apoptosis recently have been identified and cloned, because the apoptotic pathways have not been delineated clearly, many novel genes and gene products involved in the processes await discovery.

One group of molecules known to play a significant role in regulating cellular development are the Wnt family of proteins. Wnts are encoded by a large gene family whose members have been found in round worms, insects, cartilaginous fishes and vertebrates. Wnts are thought to function in a variety of developmental and physiological processes since many diverse species have multiple conserved Wnt genes (McMahon, Trends Genet. (1992) 8:236-242; and Nusse & Varmus, Cell (1992) 69:1073-1087).

Wnt genes encode secreted glycoproteins that are thought to function as paracrine or autocrine signals active in several primitive cell types (McMahon (1992) and Nusse & Varmus (1992), supra). The Wnt growth factor family includes more than 10 genes in the mouse (Wnt-1, 2, 3a, 3b, 4, 5a, 5b, 6, 7a 7b, 8a, 8b, 10b, 11, 12) (see, e.g., Gavin et al., Genes Dev. (1990) 4: 2319-2332; Lee et al., Proc. Natl. Acad. Sci. USA (1995) 92:2268-2272; and Christiansen et al., Mech. Dev. (1995) 51:341-350) and at least 7 genes in human (Wnt-1, 2, 3,4, 5a, 7a and 7b) (see, e.g., Vant Veer et al., Mol. Cell. Biol. (1984) 4:2532-2534).

Identification of Wnt receptors was hampered by the relative insolubility of the Wnt proteins, which tend to remain tightly bound to cells or the extracellular matrix. However, several observations now indicate that members of the Frizzled (FZ) family of molecules can function as receptors for Wnt proteins or as components of a Wnt receptor complex (He et al., Science (1997) 275:1652-1654).

Each member of the FZ receptor gene family encodes an integral membrane protein with a large extracellular portion, seven putative transmembrane domains and a cytoplasmic tail, see e.g., Wang et al., J. Biol. Chem. (1997) 271:468-76). Near the NH₂-terminus of the extracellular portion is a cysteine-rich domain (CRD) that is well conserved among other members of the FZ family. The CRD, comprised of about 110 amino acid residues, including 10 invariant cysteines, is the putative binding site for Wnt ligands (Bbanot et al., Nature (1996) 382:25-30). There are 10 known genes in the FZ family of receptors.

Most Wnt-FZ signals are mediated through inhibition of glycogen synthase kinase (GSK3 β) and accumulation of β-catenin in the nucleus. β-catenin activates c-myc which can lead to apoptosis in some cells. Thus, Wnt signaling through FZ1 and FZ2 and maintenance of β-catenin can lead to cell death, especially in immature cells in the cerebellum. Further, overexpression of FZ1 and FZ2, and of β-catenin can induce apoptosis. However, some Wnt-FZ signaling pathways are β-catenin independent.

Ultimately, Wnt transmits its signal by allowing β-catenin to accumulate in the cell cytoplasm. There, β-catenin binds to members of the Tcf-Lef transcription factor family and translocates to the nucleus. When Wnt is absent, β-catenin instead forms a complex with GSK3 and the adenomatous polyposis coli (APC) tumor suppressor protein. That interaction is associated with the phosphorylation of β-catenin, marking it for ubiquitination and degradation. Wnt permits the accumulation of β-catenin by inhibiting the function of GSK3.

The existence of molecules that have a FZ CRD but lack the seven transmembrane motif and cytoplasmic tail suggested that there was a subfamily of proteins that function as regulators of Wnt activity. Soluble frizzled related proteins (SFRPs), for example, the nucleic acid sequence leaving accession number AF056087, are related to the secreted apoptosis related proteins (SARPs) and comprise a family of secreted molecules that contain a CRD domain highly homologous to the FZ CRD (Finch et al., Proc. Natl. Acad. Sci. USA (1997) 94:6770-6775). SARPs block Wnt signaling by interacting with Wnt or by forming nonfunctional homomeric complexes with membrane bound FZ.

The disregulation of Wnt pathways appears to be a factor in aberrant growth as well as in development. Given the potential complexity of interactions between the multiple members of the Wnt and FZ families, additional mechanisms might exist to modulate Wnt regulated events (e.g., apoptosis) during specific periods of development or in certain tissues during disease development/injury. The identification of such mechanisms and in particular, the effectors of those mechanisms are important for understanding and modulating the processes of cellular regulation.

Free Radical Neurotoxicity

Nitric oxide (NO) is a widespread and multifunctional biological messenger molecule. NO may play a role not only in physiologic neuronal functions, such as neurotransmitter release, neural development, regeneration, synaptic plasticity and regulation of gene expression, but also in a variety of neurological disorders in which excessive production of NO leads to neural injury (Yun et al., Mol Psychiatr (1997) 2:300-310).

NO is formed when L-arginine is oxidized to citrulline by the action of the enzyme nitric oxide synthase (NOS). Although NO itself is a free radical having an unpaired electron, it is not felt to participate in any significantly damaging chemical reactions in and of itself. However, when reacting with superoxide anion, the extremely reactant and potent oxidant, peroxynitrite (ONOO⁻) is formed (<www.gsdl.com/news/1999/1990302/index>, last visited 12 Nov. 2002).

N-methyl-D-aspartate (NMDA) receptor-mediated neurotoxicity may depend, in part, on the generation of peroxynitrite (OONO⁻) via NO (Lipton et al., Nature (1993) 364(6438):626-632). That form of neurotoxicity is thought to contribute to a final common pathway of injury in a wide variety of acute and chronic neurologic disorders, including focal ischemia, trauma, epilepsy, Huntington's disease, Alzheimer's disease, amyotrophic lateral sclerosis, AIDS dementia and other neurodegenerative diseases (Bonfoco et al., Proc. Natl. Acad. Sci. USA (1995) 92:7162-7166). Further, peroxynitrite has been implicated in a variety of damaging intraneuronal events including DNA strand breaks, DNA deamination, nitration of proteins including superoxide dismutase and damage to mitochondrial complex I (www.gsdl.com/news/1999/1990302/index, last visited 12 Nov. 2002). Indeed, ONOO⁻ has been shown to cause neuronal death. It has been proposed that such neuronal death occurs in different disorders of the CNS such as brain ischemia, AIDS-associated dementia, amyothrophic lateral sclerosis etc. (Moro et al., Neuropharmacology (1998) 37(8):1071-1079). Moreover, excess glutamate acting via NMDA receptors mediates cell death in focal cerebral ischemia (Yun et al. (1997), supra). Glutamate neurotoxicity also may play a part in neurodegenerative diseases such as Huntington's disease and Alzheimer's disease (Yun et al. (1997) supra).

Thus, depending on the insult, NMDA or nitric oxide/superoxide can result in apoptotic neuronal cell damage.

NMDA receptor-mediated death has been shown to be enhanced by coculture of cerebral granular cells (CGC) with immunostimulated microglia cells (Hewett et al., Neuron (1994) 13(2):487-494; Kim et al., J. Neurosci. Res. (1998) 54(1):17-26), thus intimating a role for inducible NOS in that type of neurotoxicity. Further, that enhancement was mimicked by the NO releaser, 3-morpholinosydnonimine (SIN-1). Moreover, such potentiation/enhancement of NMDA neurotoxicity and the enhancement mimicked by NO generators (e.g. SIN-1 or S-nitroso-N-acetylrpenicillamine: SNAP) seem to be blocked by NOS inhibition or antioxidants (superoxide dismutase/catalase) (Hewett et al. (1994) supra; Kim et al. (1998) supra).

In contrast, treatment of CGCs with NOS inhibitors was unable to rescue such cells after exposure to ceramide (Monti et al., Neurochem. Int. (2001) 39(1): 11-18; Nagano et al., J. Neurochem. (2001) 77(6):1486-1495). Further, apoptosis observed with exposure to ceramide may not involve the action of NMDA receptors (Centeno et al., Neuroreport (1998) 9(18):4199-4203; Moore et al., Br. J. Pharmacol. (2002) 135(4):1069-1077).

Taken together, the data suggest that the action of ceramnide is not primarily dependent on NO production and that ceramide and NO generators such as SIN-1 or SNAP induce apoptosis through separate pathways.

Thus, given the number of disease associated with the NMDA/peroxynitrite (supra), molecules selective for rescuing cells exposed to SIN-1, like neurotoxins, should be valuable as selective anti-apoptosis agents and useful in effective treatment modalities where NMDA/peroxynitrite is associated with neurological disease.

Applicants have identified a Secreted Neural Apoptosis Inhibiting Protein (SNAIP) that is neuroprotective and selectively protects against, for example, SIN-1, but not C2 ceramide, neurotoxicity.

### SUMMARY OF THE INVENTION

The instant invention relates to a method of modulating peroxynitrite induced apoptosis in neuronal cells comprising contacting said cells with secreted neural apoptosis inhibiting proteins (SNAIP). In a related aspect the method comprises the addition of heparin. In another related aspect, the method modulates glutamate/NMDA-induced apoptosis.

The invention also relates to apoptotic pathways comprising induction of selected genes including p38 MAPK and growth arrest and DNA damage-inducible genes (i.e., GADDs).

Further, the instant invention relates to a method of protecting neurons from peroxynitrite-associated free radical-mediated cell death comprising contacting said cells with a SNAIP.

Moreover, the invention relates to a method of determining neuroprotective genomic targets associated with the peroxynitrite toxicity pathway. In a related aspect, such a method may include the steps of contacting neuronal cells with and without a SNAIP, contacting said cells with a peroxynitrite inducer, determining modulation of gene expression in exposed cells and identifying genes that are modulated in the presence or absence of a SNAIP and the inducer. Such a method is envisaged to identify genes and correlate such genes with inhibition of apoptosis induced by the actions of peroxynitrite induction. In a related aspect, said method also comprises contacting cells with heparin. In a further related aspect, the inducer is SIN-1.

The instant invention also relates to a method for treating neuronal diseases associated with free radical-mediated cell death comprising administering to a patient in need thereof, a therapeutically effective amount of a SNAIP, where cell death is apoptosis. In a related aspect, diseases associated with apoptosis include Parkinson's disease, multiple sclerosis, focal cerebral ischemia, AIDS-associated dementia, amyothrophic lateral sclerosis, spinal cord injury, traumatic brain injury, stroke and Alzheimer's disease. In a related aspect, the treatment modality includes administration of heparin.

In another aspect of the invention, therapeutic methods are disclosed for modulating SNAIP expression, including administration of peptides, agonists, antagonists, inverse agonists and/or antibody to a patient in need thereof. Also, a SNAIP can be used for identifying molecules that bind FZ. Those molecules can be agonists, antagonists, merely engage FZ, but preferably an antagonist to minimize Wnt signaling to avoid apoptosis.

In another aspect of the invention, methods are disclosed for identifying modulators of a SNAIP comprising the steps of providing a chemical moiety, providing a cell expressing a SNAIP or purified a SNAIP and determining whether the chemical moiety binds a SNAIP. In a related aspect, the chemical moieties can include, but are not limited to, peptides, antibodies, and small molecules.

Another aspect of the invention includes therapeutic compositions, where such compositions include nucleic acids, antibodies, polypeptides, agonists, inverse agonists and antagonists. Further, methods of the invention also include methods of treating disease states by administering such therapeutic compositions to a patient in need thereof. The active agent can be a molecule identified using a SNAIP or a SNAIP per se.

Those and other aspects of the invention will become evident on reference to the following detailed description and the attached drawings. In addition, various references are set forth herein which describe in more detail certain procedures or compositions. Each of those references hereby is incorporated herein by reference in entirety as if each were individually noted for incorporation.

### DETAILED DESCRIPTION OF THE INVENTION

The protein of the instant invention is approximately 60% identical in homology to a family of proteins called secreted apoptotic related proteins (SARPs). Applicants have localized expression of the molecule in the brain where it appears in higher abundance in fetal than in adult brain. The protein has been identified in the adult forebrain and midbrain and the posterior eye region but not in the area in which it was discovered, the ventricular zone. There has been no detailed association between the protein and any given cell type. The protein appears to be anti-apoptotic. SNAIP protects neurons from free radical mediated cell death.

Thus, in one embodiment, a SNAIP and its regulation are targets for drug discovery for therapeutic intervention in neurodegenerative diseases to include, but not limited to, Parkinson's disease, multiple sclerosis, focal cerebral ischemia, AIDS-associated dementia, amyothrophic lateral sclerosis, spinal cord injury, traumatic brain injury, stroke and Alzheimer's disease.

Deregulated excess generation of NO can initiate a neurotoxic cascade. NO presumably kills neurons via peroxynitrite. That powerful oxidant is thought to be involved in most NO-mediated neurotoxicity. Peroxynitrite further may decompose to hydroxyl and nitrogen dioxide radicals which also are highly reactive and biologically destructive leading to a variety of neurological disorders arising from excessive production of NO.

For example, neuronally-derived NO plays an important role in mediating neuronal cell death following focal ischemia. In the late stages of cerebral ischemia (>6 h), post-ischemic inflammation induces iNOS expression, and the sustained generation of large amounts of NO leads to delayed neural injury (Yun et al. (1997) supra).

In a related aspect, 3-nitrotyrosine (3-NT) is a specific marker of protein nitration by peroxynitrite (ONOO⁻) produced from NO and superoxide. Increase in 3-NT-containing protein (3-NT protein) was reported in brains from patients with some neurodegenerative disorders (Yamamoto et al., J. Neural. Transm. (2002) 109(1):1-13). Thus, in one embodiment, 3-NT is used as a marker to identify pathways associated with a SNAIP.

In a further related aspect, activation of a mitogen-activated protein kinase (MAPK) pathway by NO may be a key to how NO regulates neuronal growth, differentiation, survival and death. Since the MAPK signaling pathways play a central role in growth factor response (ERK) or stress response (JNK, p38 MAPK) in the nervous system, NO-MAPK signaling may underlie NO's role in neuronal survival, differentiation and apoptotic cell death during neuronal development and disease/injury (Yun et al. (1997) supra).

A peroxynitrite generator, 3-morpholinosydonimine (SIN-1), was found to induce the expression of three different growth arrest and DNA damage-inducible (GADD) mRNAs, GADD34, GADD45, and GADD153, at the early phase during cell death in human neuroblastoma SH-SY5Y cells. Peroxynitrite also activated p38 MAPK. The expression of three GADD genes and also p38 MAPK phosphorylation were suppressed by treatment with radical scavengers, superoxide dismutase plus catalase and glutathione (Ohashi et al., Free Radic. Biol. Med. (2001) 30(2):213-221). Thus, in one embodiment, the pathway of interest comprises GADD34, GADD45, GADD153 and p38 MAPK.

SNAIP is neuroprotective and selectively protects against SIN-1, but not C2 ceramide, neurotoxicity. SNAIP is released by cells into the medium, particularly in the presence of heparin.

In a related aspect, NO generators such as SIN-1 or SNAP and ceramide induce apoptosis through separate pathways. In a preferred embodiment, SNAIP selectively protects against NMDA-induced apoptosis.

The presence of a SNAIP in those and other tissues suggests SNAIP is involved in a variety of nervous system disease states involving various neurodegenerative disorders. Identification of a SNAIP in those tissues and cloning of the gene encoding a SNAIP provides a variety of therapeutic approaches to regulate SNAIP expression and activity so as to provide therapeutic approaches to treating diseases involving SNAIP.

Human SNAIP bears only 60% amino acid identity to and is not related to the secreted apoptosis related protein (SARP) family of molecules having certain conserved structural and functional features. The term “family,” when referring to the protein and nucleic acid molecules of the invention, is intended to mean two or more proteins or nucleic acid molecules having an overall common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family also may have common functional characteristics.

The term “equivalent amino acid residues” herein means the amino acids occupy substantially the same position within a protein sequence when two or more sequences are aligned for analysis.

The term “sufficiently identical” is used herein to refer to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 75% identity, preferably 80% identity, more preferably 85%, 95% or 98% identity are defined herein as sufficiently identical.

As used interchangeably herein, a “SNAIP activity”, “biological activity of SNAIP” or “functional activity of SNAIP”, refers to an activity exerted by a SNAIP protein, polypeptide or nucleic acid molecule on a SNAIP responsive cell according to standard techniques or as taught herein. A SNAIP activity can be a direct activity, such as an association with a second protein, or an indirect activity, such as a cellular signaling activity mediated by interaction of a SNAIP protein with a second protein. In a preferred embodiment, a SNAIP activity includes at least one or more of the following activities: (i) the ability to interact with proteins in the Wnt/FZ signaling pathway; (ii) the ability to interact with a SNAIP receptor (e.g., FZ); (iii) the ability to interact with an intracellular target protein; and (iv) the ability to induce a SNAIP biological manifestation. For example, a SNAIP activity or manifestation includes, but is not limited to, inhibiting the binding of Wnt to FZ as may be determined by means well known in the art.

Accordingly, another embodiment of the invention features isolated SNAIP proteins and polypeptides having a SNAIP activity.

Various aspects of the invention are described in further detail in the following subsections.

I. Isolated Nucleic Acid Molecules

One aspect of the invention pertains to isolated nucleic acid molecules that encode SNAIPs or biologically active portions thereof; as well as nucleic acid molecules sufficient for use as hybridization probes to identify SNAIP-encoding nucleic acids (e.g., SNAIP mRNA) and fragments for use as PCR primers for the amplification or mutation of SNAIP nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

An “isolated” nucleic acid molecule is one that is separated from other nucleic acid molecules that are present in the natural source of the nucleic acid. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated SNAIP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium, when produced by recombinant techniques, or substantially free of chemical precursors or other chemicals when chemically synthesized.

A nucleic acid molecule of the instant invention or a complement of any of those nucleotide sequences, can be isolated using standard molecular biology techniques (e.g., as described in Sambrook et al., eds., “Molecular Cloning: A Laboratory Manual,” 2nd ed., Cold Spring Harbor Laboratory Press, Cold Spring Harbor, N.Y., 1989).

The nucleotide sequence determined from the cloning of the human SNAIP gene allows for the generation of probes and primers designed for use in identifying and/or cloning SNAIP homologues in other cell types, e.g., from other tissues, as well as SNAIP homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SNAIP or of a naturally occurring mutant of SNAIP. Probes based on the human SNAIP nucleotide sequence can be used to detect transcripts or genomic sequences encoding the similar or identical proteins. The probe may comprise a label group attached thereto, e.g., a radioisotope, a fluorescent compound, an enzyme or an enzyme co-factor. Such probes can be used as part of a diagnostic test kit for identifying cells or tissues which improperly express a SNAIP protein, such as by measuring levels of SNAIP-encoding nucleic acids in a sample of cells from a subject, e.g., detecting SNAIP mRNA levels or determining whether a genomic SNAIP gene has been mutated or deleted.

A nucleic acid fragment encoding a “biologically active portion of SNAIP” can be prepared by isolating a polynucleotide which encodes a polypeptide having a SNAIP biological activity (e.g., inhibiting apoptosis), expressing the encoded portion of SNAIP protein (e.g., by recombinant expression) and assessing the activity of the encoded portion of SNAIP. Alternatively, the fragment may bind to an antibody known to neutralize SNAIP activity.

One of skill in the art will appreciate that DNA sequence polymorphisms that lead to changes in the amino acid sequences of SNAIP may exist within a population (e.g., the human population). Such genetic polymorphism in the SNAIP gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a SNAIP protein, preferably a mammalian SNAIP protein. As used herein, the phrase “allelic variant” refers to a nucleotide sequence that occurs at a SNAIP locus or to a polypeptide encoded by the nucleotide sequence, wherein the nucleotide or polypeptide is not the prevalent form found in a given population. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in SNAIP that are the result of natural allelic variation and that do not alter the functional activity of SNAIP are intended to be within the scope of the invention.

Moreover, nucleic acid molecules encoding SNAIP proteins from other species (SNAIP homologues), which have a nucleotide sequence which differs from that of a human SNAIP, are intended to be within the scope of the invention. Nucleic acid molecules corresponding to natural allelic variants and homologues of the SNAIP cDNA of the invention can be isolated based on identity to the human SNAIP nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 300, 325, 350, 375, 400, 425, 450, 500, 550, 600, 650, 700, 800, 900, 1000 or 1100 nucleotides in length and hybridizes under stringent conditions to a nucleic acid molecule with SNAIP activity.

As used herein, the term “hybridizes under stringent conditions” is intended to describe conditions for hybridization and washing under which nucleotide sequences at least 60% (65%, 70% and preferably 75% or greater) identical to each other typically remain hybridized to each other. Such stringent conditions are known to those skilled in the art and can be found, for example, in “Current Protocols in Molecular Biology,” John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2×SSC, 0.1% SDS at 50-65° C. As used herein, a “naturally-occurring” nucleic acid molecule refers to an RNA or DNA molecule having a nucleotide sequence that occurs in nature (e.g., encodes a naturally occurring protein).

In addition to naturally-occurring allelic variants of the SNAIP sequence that may exist in the population, the skilled artisan further will appreciate that changes can be introduced by mutation thereby leading to changes in the amino acid sequence of the encoded SNAIP protein, without altering the biological activity of the SNAIP protein. For example, one can make nucleotide substitutions leading to amino acid substitutions at “non-essential” amino acid residues. A “non-essential” amino acid residue is a residue that can be altered from the wild-type sequence of SNAIP without altering the biological activity, whereas an “essential” amino acid residue is required for biological activity. For example, amino acid residues that are not conserved or only semi-conserved among SNAIP of various species may be non-essential for activity and thus would be likely targets for alteration. Alternatively, amino acid residues that are conserved among the SNAIP proteins of various species may be essential for activity and thus would not be likely targets for alteration.

Accordingly, another aspect of the invention pertains to nucleic acid molecules encoding SNAIP proteins that contain changes in amino acid residues that are not essential for activity. In one embodiment, the isolated nucleic acid molecule includes a nucleotide sequence encoding a protein that includes an amino acid sequence that is at least about 87% identical, 90%, 93%, 95%, 98% or 99% identical to a polypeptide with SNAIP activity.

An isolated nucleic acid molecule encoding a SNAIP protein having a variant sequence can be created by introducing one or more nucleotide substitutions, additions or deletions into the nucleotide sequence of a naturally occurring SNAIP such that one or more amino acid substitutions, additions or deletions are introduced into the encoded protein.

Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Those families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine and cysteine), nonpolar side chains (e.g., alanine, valine, leucine, isoleucine, proline, phenylalanine, methionine and tryptophan), beta-branched side chains (e.g., threonine, valine and isoleucine) and aromatic side chains (e.g., tyrosine, phenylalanine, tryptophan and histidine). Thus, a predicted nonessential amino acid residue in SNAIP is preferably replaced with another amino acid residue from the same side chain family. Alternatively, mutations can be introduced randomly along all or part of a SNAIP coding sequence, such as by saturation mutagenesis, and the resultant mutants can be screened for SNAIP biological activity to identify mutants that retain activity. Following mutagenesis, the encoded protein can be expressed recombinantly and the activity of the protein can be determined.

In a preferred embodiment, a mutant SNAIP protein can be assayed for: (1) the ability to form protein:protein interactions with proteins in the SNAIP signaling pathway; (2) the ability to bind a SNAIP receptor (e.g., FZ); or (3) the ability to bind to an intracellular target protein. In yet another preferred embodiment, a mutant SNAIP can be assayed for the ability to modulate cellular proliferation or cellular differentiation.

The instant invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire SNAIP coding strand, or to only a portion thereof, e.g., all or part of the protein coding region (or open reading frame). An antisense nucleic acid molecule can be antisense to a noncoding region of the coding strand of a nucleotide sequence encoding SNAIP. The noncoding regions (“5′ and 3′ untranslated or flanking regions”) are the 5′ and 3′ sequences that flank the coding region and are not translated into amino acids. An antisense molecule can be used to inhibit FZ expression, for example.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be synthesized chemically using naturally occurring nucleotides or variously modified nucleotides designed to increase the biological stability of the molecules or to increase the physical stability of the duplex formed between the antisense and sense nucleic acids, e.g., phosphorothioate derivatives, phosphonate derivatives and acridine-substituted nucleotides can be used.

The instant invention also contemplates other inhibiting RNA molecules, such as RNAi molecules. Appropriate double-standard or hairpin RNA's are configured and used to modulate SNAIP production.

Examples of modified nucleotides which can be used to generate the antisense and other nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 1-methylguanine, 5-carboxymethylaminomethyl-2-thiouridine, 5-carboxymethylaminomethyluracil, dihydrouracil, β-D-galactosylqueosine, inosine, N⁶-isopentenyladenine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N⁶-isopentenyladenine, uracil-5-oxyacetic acid, butoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

An antisense nucleic acid or other nucleic acid molecule of the invention can be an α-anomeric nucleic acid molecule. An α-anomeric nucleic acid molecule forms specific double-stranded hybrids with complementary RNA in which the strands run parallel to each other (Gaultier et al., Nucleic Acids Res. (1987) 15:6625-6641). The antisense nucleic acid or other nucleic acid molecule also can comprise a methylribonucleotide (Inoue et al., Nucleic Acids Res (1987) 15:6131-6148) or a chimeric RNA-DNA analogue (Inoue et al., FEBS Lett. (1987) 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff et al., Nature (1988) 334:585-591)) can be used to cleave catalytically SNAIP mRNA transcripts thereby to inhibit translation of SNAIP mRNA. A ribozyme having specificity for a SNAIP-encoding nucleic acid can be designed based on the nucleotide sequence of a naturally occurring SNAIP cDNA. For example, a derivative of a Tetrahymena L-19 IVS RNA can be constructed in which the nucleotide sequence of the active site is complementary to the nucleotide sequence to be cleaved in a SNAIP-encoding mRNA, see, e.g., Cech et al., U.S. Pat. No. 4,987,071; and Cech et al., U.S. Pat. No. 5,116,742. Alternatively, SNAIP mRNA can be used to select a catalytic RNA having a specific ribonuclease activity from a pool of RNA molecules, see, e.g., Bartel et al., Science (1993) 261:1411-1418.

The invention also encompasses nucleic acid molecules that form triple helical structures. For example, SNAIP gene expression can be inhibited by targeting nucleotide sequences complementary to the regulatory region of the SNAIP (e.g., the SNAIP promoter and/or enhancers) to form triple helical structures that prevent transcription of the SNAIP gene in target cells, see generally Helene, Anticancer Drug Dis. (1991) 6(6):569; Helene, Ann. N.Y. Acad. Sci. (1992) 660:27; and Maher, Bioassays (1992) 14(12):807.

In preferred embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (See Hyrup et al., Bioorganic & Medicinal Chemistry (1996) 4:5). As used herein, the terms “peptide nucleic acids” or “PNAs” refer to nucleic acid mimics, e.g., DNA mimics, in which the deoxyribose phosphate backbone is replaced by a pseudonucleotide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al., Proc. Natl. Acad. Sci. USA (1996) 93:14670.

PNAs of SNAIP can be used in therapeutic and diagnostic applications. For example, PNAs can be used as antisense or antigene agents for sequence-specific modulation of gene expression by, e.g., inducing transcription or translation arrest or inhibiting replication. PNAs of SNAIP also can be used, e.g., in the analysis of single base pair mutations in a gene by, e.g., PNA-directed PCR clamping; as artificial restriction enzymes when used in combination with other enzymes, e.g., S1 nucleases (Hyrup (1996) supra) or as probes or primers for DNA sequence and hybridization (Hyrup (1996) supra; Perry-O'Keefe et al. (1996) supra).

In another embodiment, PNAs of a SNAIP can be modified, e.g., to enhance stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al., Nucleic Acids Res. (1996) 24(17):3357-63; Mag et al., Nucleic Acids Res. (1989) 17:5973; and Peterser et al., Bioorganic Med. Chem. Lett. (1975) 5:1119.

II. Isolated SNAIP Proteins and Anti-SNAIP Antibodies

One aspect of the invention pertains to isolated SNAIP proteins, and biologically active portions thereof, as well as polypeptide fragments suitable, for example, for use as immunogens to raise anti-SNAIP antibodies. In one embodiment, native SNAIP proteins can be isolated from cells or tissue sources by an appropriate purification scheme using standard protein purification techniques. In another embodiment, SNAIP proteins are produced by recombinant DNA techniques. Alternative to recombinant expression, a SNAIP protein or polypeptide can be synthesized chemically using standard peptide synthesis techniques.

An “isolated” or “purified” protein or biologically active portion thereof is substantially free of cellular material or other contaminating proteins from the cell or tissue source from which the SNAIP protein is derived, or substantially free of chemical precursors or other chemicals when chemically synthesized. The language “substantially free of cellular material” includes preparations of SNAIP protein in which the protein is separated from cellular components of the cells from which it is isolated or recombinantly produced. Thus, SNAIP protein that is substantially free of cellular material includes preparations of SNAIP protein having less than about 30%, 20%, 10% or 5% (by dry weight) of non-SNAIP protein (also referred to herein as a “contaminating protein”). When the SNAIP protein or biologically active portion thereof is produced recombinantly, it is also preferably substantially free of culture medium, i.e., culture medium represents less than about 20%, 10% or 5% of the volume of the protein preparation. When SNAIP protein is produced by chemical synthesis, it is preferably substantially free of chemical precursors or other chemicals, i.e., it is separated from chemical precursors or other chemicals which are involved in the synthesis of the protein. Accordingly, such preparations of SNAIP protein have less than about 30%, 20%, 10% or 5% (by dry weight) of chemical precursors or non-SNAIP chemicals.

Biologically active portions of a SNAIP protein include peptides comprising amino acid sequences sufficiently identical to or derived from the amino acid sequence of the SNAIP protein which include fewer amino acids than the full length SNAIP proteins, and exhibit at least one activity of a SNAIP protein. Typically, biologically active portions comprise a domain or motif with at least one activity of the SNAIP protein. A biologically active portion of a SNAIP protein can be a polypeptide that is, for example, 10, 25, 50, 100 or more amino acids in length. Preferred biologically active polypeptides include one or more identified SNAIP structural domains, e.g., the one or more extracellular domains thereof.

Moreover, other biologically active portions, in which other regions of the protein are deleted, can be prepared by recombinant techniques and evaluated for one or more of the functional activities of a native SNAIP protein.

Accordingly, a useful SNAIP protein is a protein which includes an amino acid sequence at least about 88%, preferably 90%, 93%, 95% or 99% identical to the amino acid sequence of the naturally occurring SNAIP and retains the functional activity of SNAIP.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions then are compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., percent identity=number of identical positions/total number of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin et al., Proc. Natl. Acad. Sci. USA (1990) 87:2264, modified as in Karlin et al., Proc. Natl. Acad. Sci. USA (1993) 90:5873-5877. Such an algorithm is incorporated into the NBLAST and XBLAST programs of Altschul et al., J. Mol. Bio. (1990) 215:403. BLAST nucleotide searches can be performed with the NBLAST program, for example, score=100, wordlength=12 to obtain nucleotide sequences homologous to SNAIP nucleic acid molecules of the invention. BLAST protein searches can be performed with the XBLAST program, for example, score=50, wordlength=3, to obtain amino acid sequences homologous to SNAIP protein molecules of the invention. To obtain gapped alignments for comparison purposes, Gapped BLAST can be utilized as described in Altschul et al., Nucleic Acids Res. (1997) 25:3389. Alternatively, PSI-Blast can be used to perform an iterated search that detects distant relationships between molecules. Altschul et al., (1997) supra. When utilizing BLAST Gapped BLAST, and PSI-Blast programs, the default parameters of the respective programs (e.g., XBLAST and NBLAST) can be used, see http://www.ncbi.nlm.nih.gov.

Another preferred, non-limiting example of a mathematical algorithm utilized for the comparison of sequences is the algorithm of Myers et al., CABIOS (1988) 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

In a preferred embodiment, the Wnt binding portion of interest is produced. That portion of SNAIP can be used alone or fused to another molecule, such as a reporter molecule using techniques and reagents known in the art. In that way, soluble SNAIP can be used to downregulate FZ by capturing Wnt prior to Wnt engaging FZ.

In certain host cells (e.g., mammalian host cells), expression and/or secretion of SNAIP can be increased through use of a heterologous signal sequence. For example, the gp6® secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

Preferably, a SNAIP chimeric or fusion protein of the invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see e.g., Ausubel et al., supra). Moreover, many expression vectors are commercially available that already encode a fusion moiety (e.g., a GST polypeptide). A SNAIP-encoding nucleic acid can be cloned into such an expression vector such that the fusion moiety is linked in-frame to the SNAIP protein.

The instant invention also pertains to variants of the SNAIP proteins (i.e., proteins having a sequence that differs from that of the naturally occurring, prevalent SNAIP allele amino acid sequence). Such variants can function as SNAIP mimetics. Variants of the SNAIP protein can be generated by mutagenesis, e.g., discrete point mutation or truncation of the SNAIP protein. An agonist or mimetic of a SNAIP protein retains substantially the same, or a subset, of the biological activities of the naturally occurring form of the SNAIP protein. Thus, specific biological effects can be elicited by treatment with a variant of limited or enhanced function. Treatment of a subject with a variant having a subset of the biological activities of the naturally occurring form of the protein can have fewer side effects in a subject relative to treatment with the naturally occurring form of the SNAIP proteins.

Variants of the SNAIP protein can be identified by screening combinatorial libraries of mutants, e.g., truncation mutants, of the SNAIP protein for SNAIP protein activity. In one embodiment, a variegated library of SNAIP variants is generated by combinatorial mutagenesis at the nucleic acid level and is encoded by a variegated gene library. A variegated library of SNAIP variants can be produced by, for example, enzymatically ligating a mixture of synthetic oligonucleotides into gene sequences such that a degenerate set of potential SNAIP sequences is expressible as individual polypeptides, or alternatively, as a set of larger fusion proteins (e.g., for phage display) containing the set of SNAIP sequences therein. There are a variety of methods that can be used to produce libraries of potential SNAIP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SNAIP sequences. Methods for synthesizing degenerate oligonucleotides are known in the art (See, e.g., Narang, Tetrahedron (1983) 39:3; Itakura et al., Ann. Rev. Biochem. (1984) 53:323; Itakura et al., Science (1984) 198:1056; Ike et al., Nucleic Acid Res. (1983) 11:477).

In addition, libraries of fragments of the SNAIP protein coding sequence can be used to generate a variegated population of SNAIP fragments for screening and subsequent selection of variants of a SNAIP protein. In one embodiment, a library of coding sequence fragments can be generated by treating a double-stranded PCR fragment of a SNAIP coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By that method, an expression library can be derived which encodes N-terminal and internal fragments of various sizes of a SNAIP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SNAIP proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify SNAIP variants (Arkin et al., Proc. Natl. Acad. Sci. USA (1992) 89:7811-7815; Delgrave et al., Protein Engineering (1993) 6(3):327-331).

An isolated SNAIP protein, or a portion or fragment thereof, can be used as an immunogen to generate antibodies that bind SNAIP using standard techniques for polyclonal and monoclonal antibody preparation. The full-length SNAIP protein can be used or, alternatively, the invention provides antigenic peptide fragments of SNAIP for use as immunogens. The antigenic peptide of SNAIP comprises at least 8 (preferably 10, 15, 20, or 30) amino acid residues of SNAIP and encompasses an epitope of SNAIP such that an antibody raised against the peptide forms a specific immune complex with SNAIP. The epitope may be attached to a carrier molecule such as albumin.

A SNAIP immunogen typically is used to prepare antibodies by immunizing a suitable subject, (e.g., rabbit, goat, mouse or other mammal) with the immunogen. An appropriate immunogenic preparation can contain, for example, recombinantly expressed SNAIP protein or a chemically synthesized SNAIP polypeptide. The preparation further can include an adjuvant, such as Freund's complete or incomplete adjuvant, or similar immunostimulatory agent. Immunization of a suitable subject with an immunogenic SNAIP preparation induces a polyclonal anti-SNAIP antibody response.

Accordingly, another aspect of the invention pertains to anti-SNAIP antibodies. The term “antibody” as used herein refers to immunoglobulin molecules and immunologically active portions of immunoglobulin molecules, i.e., molecules that contain an antigen binding site that specifically binds SNAIP. A molecule that specifically binds to SNAIP is a molecule that binds SNAIP but does not substantially bind other molecules in a sample, e.g., a biological sample, which naturally contains SNAIP. Examples of immunologically active portions of immunoglobulin molecules include F_{(ab)} and F_{(ab′)2} fragments which can be generated by treating the antibody with an enzyme such as pepsin. The invention provides polyclonal and monoclonal antibodies that bind SNAIP. The term “monoclonal antibody” or “monoclonal antibody composition”, as used herein, refers to a population of antibody molecules that contain only one species of an antigen binding site capable of immunoreacting with a particular epitope of SNAIP. A monoclonal antibody composition thus typically displays a single binding affinity for a particular SNAIP protein with which it immunoreacts.

Polyclonal anti-SNAIP antibodies can be prepared as described above by immunizing a suitable subject with a SNAIP immunogen. The anti-SNAIP antibody titer in the immunized subject can be monitored over time by standard techniques, such as with an enzyme linked immunosorbent assay (ELISA) using immobilized SNAIP.

If desired, the antibody molecules directed against SNAIP can be isolated from the mammal (e.g., from the blood) and further purified by well-known techniques, such as protein A chromatography to obtain the IgG fraction. At an appropriate time after immunization, e.g., when the anti-SNAIP antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kohler et al., Immunol. Today (1983) 4:72), the EBV-hybridoma technique (Cole et al., Monoclonal Antibodies and Cancer Therapy, (1985), Alan R. Liss, Inc., pp. 77-96) or trioma techniques. The technology for producing hybridomas is well known (See generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with a SNAIP immunogen as described above, and the culture supernatants of the resulting hybridoma cells are screened to identify a hybridoma producing a monoclonal antibody that binds SNAIP.

Any of the many well known protocols used for fusing lymphocytes and immortalized cell lines can be applied for the purpose of generating an anti-SNAIP monoclonal antibody (see, e.g., Current Protocols in Immunology, supra; Galfre et al., Nature (1977) 266:550-552; Kenneth, in Monoclonal Antibodies: A New Dimension In Biological Analyses, Plenum Publishing Corp., New York, N.Y. (1980); and Lerner, Yale J. Biol. Med. (1981) 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/l-Ag4-1, P3-x63-Ag8.653 or Sp2/O-Ag14 myeloma lines. Those myeloma lines are available from ATCC. Typically, HAT-sensitive mouse myeloma cells are fused to mouse splenocytes using polyethylene glycol (“PEG”). Hybridoma cells resulting from the fusion then are selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind SNAIP, e.g., using a standard ELISA assay.

Alternative to preparing monoclonal antibody-secreting hybridomas, a monoclonal anti-SNAIP antibody can be identified and isolated by screening a recombinant combinatorial immunoglobulin library (e.g., an antibody phage display library) with SNAIP to thereby isolate immunoglobulin library members that bind SNAIP. Kits for generating and screening phage display libraries are commercially available (e.g., the Pharmacia Recombinant Phage Antibody System, Catalog No. 27-9400-01; and the Stratagene SurfZAP®Phage Display Kit, Catalog No. 240612).

Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology (1991) 9:1370-1372; Hay et al., Hum. Antibod. Hybridomas (1992) 3:81-85; Huse et al., Science (1989) 246:1275-1281; and Griffiths et al., EMBO J. (1993) 25 12:725-734.

Additionally, recombinant anti-SNAIP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; Europe Patent Publication No. 184,187; Europe Patent Publication No. 171,496; Europe Patent Publication No. 173,494; PCT Publication No. WO 86/01533; U.S. Pat. No. 4,816,567; Europe Patent Publication No. 125,023; Better et al., Science (1988) 240:1041-1043; Liu et al., Proc. Natl. Acad. Sci. USA (1987) 84:3439-3443; Lin et al., J. Immunol. (1987) 139:3521-3526; Sun et al., Proc. Natl. Acad. Sci. USA (1987) 84:214-218; Nishimura et al., Canc. Res. (1987) 47:999-1005; Wood et al., Nature (1985) 314:446-449; Shaw et al., J. Natl. Cancer. Inst. (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J. Immunol. (1988) 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of SNAIP. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such an epitope, e.g., an antibody that inhibits SNAIP activity, is identified. The heavy chain and the light chain of the non-human antibody are cloned and used to create phage display Fab fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria that express the non-human heavy chain. The resulting progeny phage display hybrid antibodies (human light chain/non-human heavy chain). The selected antigen is used in a panning screen to select phage which bind the selected antigen. Several rounds of selection may be required to identify such phage. Next, human light chain genes are isolated from the selected phage which bind the selected antigen. These selected human light chain genes are then used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage which bind the selected antigen. The phage selected in that step display a completely human antibody that recognizes the same epitope recognized by the original selected, non-human monoclonal antibody. The genes encoding both the heavy and light chains are isolated readily and can be manipulated further for production of human antibody. The technology is described by Jespers et al. (Bio/Technology (1994) 12:899-903).

An anti-SNAIP antibody (e.g., monoclonal antibody) can be used to isolate SNAIP by standard techniques, such as affinity chromatography or immunoprecipitation. An anti-SNAIP antibody can facilitate the purification of natural SNAIP from cells and of recombinantly produced SNAIP expressed in host cells. Moreover, an anti-SNAIP antibody can be used to detect SNAIP protein (e.g., in a cellular lysate or cell supernatant) to evaluate the abundance and pattern of expression of the SNAIP protein. Anti-SNAIP antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials and radioactive materials. Examples of suitable enzymes include horseradish peroxidase, alkaline phosphatase, galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, given fluorescent protein or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H.

SNAIP molecules can be analyzed, for example, by x-ray crystallography, to discern the structure, for example, of that portion that binds Wnt. With that structural information, the artisan can construct synthetil molecules that bind Wnt. Such SNAIP mimics can be made from any of a variety of building blocks including amino acids, nucleotides, sugars, organic molecules and the like, and combinations thereof.

SNAIP molecules also can be used as immunogens to raise antibodies that have conformations that mimic Wnt. Such antibodies, similar to antibodies raised directly to FZ, would bind FZ and would preclude binding of Wnt to FZ. Preferably such antibodies do not trigger activation of FZ.

III. Recombinant Expression Vectors and Host Cells

Another aspect of the invention pertains to vectors, preferably expression vectors, containing a nucleic acid encoding SNAIP (or a portion thereof). As used herein, the term “vector” refers to a nucleic acid molecule capable of transporting another nucleic acid to which it has been linked. One type of vector is a “plasmid”, which refers to a circular double-stranded DNA loop into which additional DNA segments can be ligated. Another type of vector is a viral vector, wherein additional DNA segments can be ligated into the viral genome because of the larger capacity of a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell on introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), that serve equivalent functions.

The recombinant expression vectors of the invention comprise nucleic acid of the invention in a form suitable for expression of the nucleic acid in a host cell. That means that the recombinant expression vectors include one or more regulatory sequences, selected on the basis of host cells to be used for expression, which is operably linked to the nucleic acid to be expressed. Within a recombinant expression vector, “operably linked” is intended to mean that the nucleotide sequence of interest is linked to the regulatory sequence(s) in a manner which allows for expression of the nucleotide sequence (e.g., in an in vivo transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology Vol. 185, Academic Press, San Diego, Calif. (1990). Regulatory sequences include those that direct constitutive expression of the nucleotide sequence in many types of host cells (e.g., tissue-specific regulatory sequences). It will be appreciated by those skilled in the art that the design of the expression vector can depend on such factors as the choice of host cell to be transformed, the level of expression of protein desired etc. The expression vectors of the invention can be introduced into host cells thereby to produce proteins or peptides, encoded by nucleic acids as described herein (e.g., SNAIP proteins, mutant forms of SNAIP, fusion proteins etc.).

The recombinant expression vectors of the invention can be designed for expression of SNAIP in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.; Smith et al., Gene (1988) 67:31-40), pMAL (New England Biolabs, Beverly, Mass.) and pRITS (Phannacia, Piscataway, N.J.) which fuse glutathione 5-transferase (GST), maltose E binding protein or protein A, respectively, to the target recombinant protein.

Examples of suitable inducible non-fusion E. coli expression vectors include pTrc (Ararm et al., Gene (1988) 69:301-315) and pET 11d (Studier et al., Gene Expression Technology: Methods in Enzymology, Academic Press, San Diego, Calif. (1990) 185:60-89). Target gene expression from the pTrc vector relies on host RNA polymerase transcription from a hybrid trp-lac fusion promoter. Target gene expression from the pET 11d vector relies on transcription from a T7 gn1-lac fusion promoter mediated by a coexpressed viral RNA polymerase (T7 gnl). That viral polymerase is supplied by host strains BL21 (DE3) or HMS 174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology, Academic Press, San Diego, Calif. (1990) 185:119-128). Another strategy is to alter the nucleic acid sequence of the nucleic acid to be inserted into an expression vector so that the individual codons for each amino acid are those preferentially utilized in E. coli (Wada et al., Nucleic Acids Res. (1992) 20:2111-2118). Such alteration of nucleic acid sequences of the invention can be carried out by standard DNA synthesis techniques.

In another embodiment, the SNAIP expression vector is a yeast expression vector. Examples of vectors for expression in yeast S. cerevisiae include pYepSecl (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.), and pPicZ (Invitrogen Corp, San Diego, Calif.).

Alternatively, SNAIP can be expressed in insect cells using baculovirus expression vectors. Baculovirus vectors available for expression of proteins in cultured insect cells (e.g., Sf9 cells) include the pAc series (Smith et al., Mol. Cell. Biol. (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors include pCDM8 (Seed, Nature (1987) 329:840) and pMT2PC (Kaufman et al., EMBO J. (1987) 6:187-195). When used in mammalian cells, the control functions of the expression vector often are provided by viral regulatory elements. For example, commonly used promoters are derived from polyoma, adenovirus 2, cytomegalovirus and simian virus 40. For other suitable expression systems for both prokaryotic and eukaryotic cells, see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev. (1987) 1:268-277), lymphoid-specific promoters (Calame et al., Adv. Immunol. (1988) 43:235-275), in particular promoters of T cell receptors (Winoto et al., EMBO J. (1989) 8:729-733) and immunoglobulins (Banerji et al., Cell (1983) 33:729-740; Queen et al., Cell (1983) 33:741-748), neuron-specific promoters (e.g., the neurofilament promoter; Byrne et al., Proc. Natl. Acad. Sci. USA (1989) 86:5473-5477), pancreas-specific promoters (Edlund et al., Science (1985) 230:912-916) and mammary gland-specific promoters (e.g., milk whey promoter; U.S. Pat. No. 4,873,316 and EPO Publication No. 264,166). Developmentally-regulated promoters also are encompassed, for example the murine hox promoters (Kessel et al., Science (1990) 249:374-379) and the α-fetoprotein promoter (Campes et al., Genes Dev. (1989) 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule that is antisense to SNAIP mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews—Trends in Genetics, Vol. 1(1)1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but still are included within the scope of the term as used herein.

A host cell can be any prokaryotic or eukaryotic cell. For example, SNAIP protein can be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection or electroporation.

For stable transfection of mammalian cells, it is known that, depending on the expression vector and transfection technique used, only a small fraction of cells may integrate the foreign DNA into the genome. To identify and to select those integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding SNAIP or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) SNAIP protein. Accordingly, the invention further provides methods for producing SNAIP protein using the host cells of the invention. In one embodiment, the method comprises culturing the host cell of invention (into which a recombinant expression vector encoding SNAIP has been introduced) in a suitable medium such that SNAIP protein is produced. In another embodiment, the method further comprises isolating SNAIP from the medium or the host cell.

The host cells of the invention also can be used to produce nonhuman transgenic animals. For example, in one embodiment, a host cell of the invention is a fertilized oocyte or an embryonic stem cell into which SNAIP-coding sequences have been introduced. Such host cells then can be used to create non-human transgenic animals in which exogenous SNAIP sequences have been introduced into the genome or homologous recombinant animals in which endogenous SNAIP sequences have been altered. Such animals are useful for studying the function and/or activity of SNAIP and for identifying and/or evaluating modulators of SNAIP activity. As used herein, a “transgenic animal” preferably is a mammal, more preferably, a rodent such as a rat or mouse, in which one or more of the cells of the animal includes a transgene. Other examples of transgenic animals include non-human primates, sheep, dogs, cows, goats, chickens, amphibians etc. A transgene is exogenous DNA which is integrated into the genome of a cell from which a taansgenic animal develops and which remains in the genome of the mature animal, thereby directing the expression of an encoded gene product in one or more cell types or tissues of the transgenic animal. As used herein, a “homologous recombinant animal” preferably is a mammal, more preferably, a mouse, in which an endogenous SNAIP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SNAIP-encoding nucleic acid into the male pronuclei of a fertilized oocyte, e.g., by microinjection, retroviral infection and allowing the oocyte to develop in a pseudopregnant female foster animal. The SNAIP cDNA sequence e.g., that of SEQ ID NO:1, can be introduced as a transgene into the genome of a non-human animal. Alternatively, a nonhuman homologue of the human SNAIP gene, such as a mouse SNAIP gene, can be isolated based on hybridization to the human SNAIP cDNA and used as a transgene. Intronic sequences and polyadenylation signals also can be included in the transgene to increase the efficiency of expression of the transgene. A tissue-specific regulatory sequence(s) can be operably linked to the SNAIP transgene to direct expression of SNAIP protein to particular cells. Methods for generating transgenic animals via embryo manipulation and microinjection, particularly animals such as mice, are conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal then can be used to breed additional animals carrying the transgene. Moreover, transgenic animals carrying a transgene encoding SNAIP further can be bred to other transgenic animals carrying other transgenes.

To create a homologous recombinant animal, a vector is prepared which contains at least a portion of a SNAIP gene (e.g., a human or a non-human homolog of the SNAIP gene, e.g., a murine SNAIP gene) into which a deletion, addition or substitution has been introduced to thereby alter, e.g., functionally disrupt, the SNAIP gene. In a preferred embodiment, the vector is designed such that, on homologous recombination, the endogenous SNAIP gene is functionally disrupted (i.e., no longer encodes a functional protein; also referred to as a “knock out” animal). Alternatively, the vector can be designed such that, on homologous recombination, the endogenous SNAIP gene is mutated or otherwise altered but still encodes functional protein (e.g., the upstream regulatory region can be altered to thereby alter the expression of the endogenous SNAIP protein). In the homologous recombination vector, the altered portion of the SNAIP gene is flanked at the 5′ and 3′ ends by additional nucleic acid of the SNAIP gene to allow for homologous recombination to occur between the exogenous SNAIP gene carried by the vector and an endogenous SNAIP gene in an embryonic stem cell. The additional flanking SNAIP nucleic acid is of sufficient length for successful homologous recombination with the endogenous gene. Typically, several kilobases of flanking DNA (both at the 5′ and 3′ ends) are included in the vector (See, e.g., Thomas et al., Cell (1987) 51:503 for a description of homologous recombination vectors). The vector is introduced into an embryonic stem cell line (e.g., by electroporation) and cells in which the introduced SNAIP gene has homologously recombined with the endogenous SNAIP gene are selected (See, e.g., Li et al., Cell (1992) 69:915). The selected cells then are injected into a blastocyst of an animal (e.g., a mouse) to form aggregation chimeras (See, e.g., Bradley in Teratocarcinomas and Embryonic Stem Cells: A Practical Approach, Robertson, ed., IRL, Oxford, (1987) pp. 113-152). A chimeric embryo then can be implanted into a suitable pseudopregnant female foster animal and the embryo brought to term. Progeny harboring the homologously recombined DNA in germ cells can be used to breed animals in which all cells of the animal contain the homologously recombined DNA by germ line transmission of the transgene. Methods for constructing homologous recombination vectors and homologous recombinant animals are described further in Bradley, Current Opinion in Bio/Technology (1991) 2:823-829 and in PCT Publication Nos. WO 90/11354, WO 91/01140, WO 92/0968 and WO 93/04169.

In another embodiment, transgenic non-human animals can be produced which contain selected systems that allow for regulated expression of the transgene. One example of such a system is the cre/loxP recombinase system of bacteriophage P1. For a description of the cre/oxP recombinase system, see, e.g., Lakso et al., Proc. Natl. Acad. Sci. USA (1992) 89:6232-6236. Another example of a recombinase system is the FLP recombinase system of S. cerevisiae (O'Gorrnan et al., Science (1991) 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein also can be produced according to the methods described in Wilmut et al., Nature (1997) 385:810-813 and PCT Publication Nos. WO 97/07668 and WO 97/07669. In brief, a cell, e.g., a somatic cell, from the transgenic animal can be isolated and induced to exit the growth cycle and enter G₀ phase. The quiescent cell then can be fused, e.g., through the use of electrical pulses, to an enucleated oocyte from an animal of the same species from which the quiescent cell is isolated. The reconstructed oocyte then is cultured such that it develops to morula or blastocyte and then transferred to a pseudopregnant female foster animal. The offspring borne of that female foster animal will be a clone of the animal from which the cell, e.g., the somatic cell, is isolated.

IV. Pharmaceutical Compositions

The SNAIP proteins, anti-SNAIP antibodies and SNAIP binding molecules (also referred to herein as “active compounds”) of the invention can be incorporated into pharmaceutical compositions suitable for administration. Such compositions typically comprise the protein or antibody and a pharmaceutically acceptable carrier, excipient or diluent. As used herein, the language, “pharmaceutically acceptable carrier,” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition of the invention is formulated to be compatible with the intended route of administration. Examples of routes of administration include parenteral, e.g., intravenous, intradermal, subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose. Acidity (pH) can be adjusted with acids or bases, such as HCl or NaOH. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL® (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SNAIP protein or anti-SNAIP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier for use as a mouthwash, wherein the compound in the fluid carrier is applied orally, swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels or creams, as generally known in the art.

The compounds also can be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparing such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited to unitary dosages, each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals. The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al., Proc. Natl. Acad. Sci. USA (1994) 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack or dispenser, together with instructions for administration.

V. Uses and Methods of the Invention

The nucleic acid molecules, proteins, SNAIP binding molecules and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials and pharmacogenomics); and d) methods of treatment (e.g., therapeutic and prophylactic). A SNAIP protein interacts with other cellular proteins and can thus be used for (i) regulation of cellular proliferation; (ii) regulation of cellular differentiation; and (iii) regulation of cell survival. The isolated nucleic acid molecules of the invention can be used to express SNAIP protein (e.g., via a recombinant expression vector in a host cell in gene therapy applications), to detect SNAIP mRNA (e.g., in a biological sample) or a genetic lesion in a SNAIP gene, and to modulate SNAIP activity (e.g., by antisense and RNAi technologies). In addition, the SNAIP proteins can be used to screen drugs or compounds which modulate or mimic the SNAIP activity or expression as well as to treat disorders characterized by insufficient or excessive production or function of SNAIP protein or production of SNAIP protein forms which have decreased or aberrant activity compared to SNAIP wild-type protein. In addition, the anti-SNAIP antibodies of the invention can be used to detect and to isolate SNAIP proteins and to modulate SNAIP activity. The invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to SNAIP proteins or have a stimulatory or inhibitory effect on, for example, SNAIP expression or SNAIP activity, or SNAIP mimics which bind Wnt or FZ and preclude binding of Wnt to FZ and preclude apoptosis.

In one embodiment, the invention provides assays for screening candidate or test compounds which bind to, modulate or mimic the activity of a SNAIP protein or polypeptide or biologically active portion thereof. The test compounds of the instant invention can be obtained using any of the numerous approaches in combinatorial library methods known in the art, including: biological libraries; spatially addressable parallel solid phase or solution phase libraries; synthetic library methods requiring deconvolution; natural product libraries; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. (1997) 12:145).

Examples of methods for the synthesis of molecular libraries can be found in the art, for example in: DeWitt et al., Proc. Natl. Acad. Sci. USA (1993) 90:6909; Erb et al., Proc. Natl. Acad. Sci. USA (1994) 91:11422; Zuckermann et al., J. Med. Chem. (1994) 37:2678; Cho et al., Science (1993) 261:1303; Carrell et al., Angew Chem. Int. Ed. Engl. (1994) 33:2059; Carell et al., Angew Chem. Int. Ed. Engl. (1994) 33:2061; and Gallop et al., J. Med. Chem. (1994) 37:1233.

Libraries of compounds may be presented in solution (e.g., Houghten, Bio/Techniques (1992) 13:412-421), or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc. Natl. Acad. Sci. USA (1992) 89:1865-1869) or phage (Scott et al., Science (1990) 249:386-390; Devlin, Science (1990) 249:404-406; Cwirla et al., Proc. Natl. Acad. Sci. USA (1990) 87:6378-6382; and Felici, J. Mol. Biol. (1991) 222:301-310).

Because a SNAIP is a ligand, SNAIP can be investigated to determine the particular portion thereof that engages, for example FZ or Wnt, practicing known methods. That particular region can be synthesized practicing known biosynthetic methods, combining carbohydrate synthesis and enzymatic reactions, for example. That portion of SNAIP is equivalent to an “epitope.” The SNAIP epitope can be modified using other monomers or non-carbohydrate moieties to yield modified epitope-carrying structures with enhanced properties, such as serum half-life, binding constant with FZ/Wnt and so on. The suitability of any one epitope variant can be determined practicing the binding and screening assays taught herein.

In one embodiment, an assay is a cell-based assay in which a cell that expresses a membrane-bound form of FZ, or a biologically active portion thereof, on the cell surface is contacted with a test compound and the ability of the test compound to competitively bind to FZ in the presence of SNAIP protein can be determined. The cell, for example, can be a yeast cell or a cell of mammalian origin. Determining the ability of the test compound to bind to the FZ can be accomplished, for example, by coupling the test compound with a radioisotope or enzymatic label such that binding of the test compound to the FZ or biologically active portion thereof can be determined by detecting the labeled compound in a complex. For example, test compounds can be labeled with ¹²⁵I, ³⁵S, ¹⁴C or ³H, either directly or indirectly, and the radioisotope detected by direct counting of radioemmnission or by scintillation counting. Alternatively, test compounds can be labeled enzymatically with, for example, horseradish peroxidase, alkaline phosphatase or luciferase, and the enzymatic label detected by determination of conversion of an appropriate substrate to product. In a preferred embodiment, the assay comprises contacting a cell which expresses a membrane-bound form of FZ, or a biologically active portion thereof, on the cell surface with a known compound which binds FZ to form an assay mixture, contacting the assay mixture with a test compound, and determining the ability of the test compound to interact with a FZ, wherein determining the ability of the test compound to interact with a FZ in the presence of SNAIP comprises determining the ability of the test compound to preferentially bind to FZ or a biologically active portion thereof as compared to SNAIP.

In yet another embodiment, an assay of the instant invention is a cell-free assay comprising contacting a SNAIP protein or biologically active portion thereof with a test compound and determining the ability of the test compound to bind to the SNAIP protein or biologically active portion thereof. Binding of the test compound to the SNAIP protein can be determined either directly or indirectly as described above. In a preferred embodiment, the assay includes contacting the SNAIP protein or biologically active portion thereof with a known compound which binds SNAIP to form an assay mixture, contacting the assay mixture with a test compound, and determining the ability of the test compound to interact with a SNAIP protein, wherein determining the ability of the test compound to interact with a SNAIP protein comprises determining the ability of the test compound to preferentially bind to SNAIP or a biologically active portion thereof, as compared to the known compound.

In another embodiment, an assay is a cell-free assay comprising contacting SNAIP protein or biologically active portion thereof with a test compound and determining the ability of the test compound to modulate (e.g., stimulate or inhibit) the activity of the SNAIP protein or a biologically active portion thereof. Determining the ability of the test compound to modulate the activity of SNAIP can be accomplished, for example, by determining the ability of the SNAIP protein to bind to a SNAIP target molecule by one of the methods described above for determining direct binding. In an alternative embodiment, determining the ability of the test compound to modulate the activity of SNAIP can be accomplished by determining the ability of the SNAIP protein to further modulate a SNAIP target molecule. For example, the catalytic/enzymatic activity of the target molecule on an appropriate substrate can be determined as described previously.

In yet another embodiment, the cell-free assay comprises contacting the SNAIP protein or biologically active portion thereof with a known compound which binds SNAIP to form an assay mixture, contacting the assay mixture with a test compound, and determining the ability of the test compound to interact with a SNAIP protein, wherein determining the ability of the test compound to interact with a SNAIP protein comprises determining the ability of the SNAIP protein to preferentially bind to or modulate the activity of a SNAIP target molecule.

In more than one embodiment of the above assay methods of the instant invention, it may be desirable to immobilize either SNAIP or Wnt to facilitate separation of complexed from uncomplexed forms of one or both of the proteins, as well as to accommodate automation of the assay. Binding of a test compound to SNAIP, or interaction of SNAIP with Wnt in the presence and absence of a candidate compound, can be accomplished in any vessel suitable for containing the reactants. Examples of such vessels include microtitre plates, test tubes and micro-centrifuge tubes. In one embodiment, a fusion protein can be provided which adds a domain that allows one or both of the proteins to be bound to a matrix. For example, glutathione-S-transferase/SNAIP fusion proteins or glutathione-S-transferase/Wnt fusion proteins can be adsorbed onto glutathione Sepharose beads (Sigma Chemical, St. Louis, Mo.) or glutathione-derivatized microtitre plates, which then are combined with the test compound or the test compound and either the non-adsorbed Wnt or SNAIP protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix and the level of SNAIP binding or activity determined using standard techniques.

Other techniques for immobilizing proteins on matrices also can be used in the screening assays of the invention. For example, either SNAIP or Wnt can be immobilized utilizing conjugation of biotin and streptavidin. Biotinylated SNAIP or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with SNAIP or Wnt but which do not interfere with binding of the SNAIP protein to a Wnt can be derivatized to the wells of the plate, and unbound Wnt or SNAIP trapped in the wells by antibody conjugation. Methods for detecting such complexes, in addition to those described above for the GST-immobilized complexes, include immunodetection of complexes using antibodies reactive with the SNAIP or target molecule, as well as enzyme-linked assays which rely on detecting an enzymatic activity associated with the SNAIP or target molecule.

In another embodiment, modulators of SNAIP expression are identified in a method in which a cell is contacted with a candidate compound and the expression of SNAIP mRNA or protein in the cell is determined. The level of expression of SNAIP mRNA or protein in the presence of the candidate compound is compared to the level of expression of SNAIP mRNA or protein in the absence of the candidate compound. The candidate compound then can be identified as a modulator of SNAIP expression based on that comparison. For example, when expression of SNAIP mRNA or protein is greater (statistically significantly greater) in the presence of the candidate compound than in its absence, the candidate compound is identified as a stimulator of SNAIP mRNA or protein expression. The level of SNAIP mRNA or protein expression in the cells can be determined by methods described herein for detecting SNAIP mRNA or protein.

In yet another aspect of the invention, the SNAIP proteins can be used as “bait proteins” in a two-hybrid assay or three hybrid assay (see, e.g., U.S. Pat. No. 5,283,317; Zervos et al., Cell (1993) 72:223-232; Madura et al., J. Biol. Chem. (1993) 268:12046-12054; Bartel et al., Bio/Techniques (1993) 14:920-924; Iwabuchi et al., Oncogene (1993) 8:1693-1696; and PCT Publication No. WO 94/10300), to identify other proteins, which bind to or interact with SNAIP (“SNAIP-binding proteins” or “SNAIP-bp”) and modulate SNAIP activity. Such SNAIP-binding proteins are also likely to be involved in the propagation of signals by the SNAIP proteins as, for example, upstream or downstream elements of the SNAIP pathway.

In yet another embodiment, the library is screened to identify SNAIP-like molecules, using, for example, a SNAIP antibody or a molecule that binds SNAIP, such as Wnt. A molecule that is bound thereby then is tested for SNAIP activity using, for example, a method as taught herein. Such a screening method reveals molecules like SNAIP that are agonists, inverse agonists or antagonists of SNAIP.

The invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, the sequences can be used to: (i) map respective genes on a chromosome and, thus, locate gene regions associated with genetic disease; (ii) identify an individual from a minute biological sample (tissue typing); and (iii) aid in forensic identification of a biological sample

The antibodies described herein can be used to detect SNAIP or FZ.

C. Predictive Medicine

The instant invention also pertains to the field of predictive medicine in which diagnostic assays, prognostic assays, pharmacogenomics and monitoring clinical trails are used for prognostic (predictive) purposes to treat thereby an individual prophylactically. Accordingly, one aspect of the instant invention relates to diagnostic assays for determining SNAIP protein and/or nucleic acid expression as well as SNAIP activity, in the context of a biological sample (e.g., blood, urine, feces, serum, cells, tissue) to determine thereby whether an individual is afflicted with a disease or disorder, or is at risk of developing a disorder, associated with aberrant or reduced SNAIP expression or activity. For example, SNAIPs are seen in vivo in areas of surviving neurons or photoreceptors following injury.

The invention also provides for prognostic (or predictive) assays for determining whether an individual is at risk of developing a disorder associated with SNAIP protein, nucleic acid expression or activity. For example, mutations in a SNAIP gene can be assayed in a biological sample. Such assays can be used for prognostic or predictive purpose to thereby prophylactically treat an individual prior to the onset of a disorder characterized by or associated with SNAIP protein, nucleic acid expression or activity.

Another aspect of the invention provides methods for determining SNAIP protein, nucleic acid expression or SNAIP activity in an individual to select thereby appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of SNAIP in clinical trials.

D. Methods of Treatment

The instant invention provides for both prophylactic and therapeutic methods of treating a subject at risk of (or susceptible to) a disorder or having a disorder associated with aberrant or reduced SNAIP expression or activity in the nervous system, and particularly, the central nervous system. Such disorders include, but are not limited to, Alzheimer's Disease and schizophrenia.

I. Prophylactic Methods

In one aspect, the invention provides a method for preventing in a subject, a disease or condition associated with an aberrant or reduced SNAIP expression or activity, by administering to the subject an agent that modulates SNAIP expression or at least one SNAIP activity. Subjects at risk for a disease that is caused or contributed to by aberrant or reduced SNAIP expression or activity can be identified by, for example, any or a combination of diagnostic or prognostic assays as described herein. Administration of a prophylactic agent can occur prior to the manifestation of symptoms characteristic of the SNAIP aberrancy, such that a disease or disorder is prevented or, alternatively, delayed in progression.

II. Therapeutic Methods

Another aspect of the invention pertains to methods of modulating SNAIP expression or activity for therapeutic purposes. The modulatory method of the invention involves contacting a cell with an agent that modulates one or more of the activities of SNAIP protein activity associated with the cell. The agent may be a mimic of SNAIP. The mimic may be a polynucleotide, polypeptide, polysaccharide, organic molecule, inorganic molecule or combinations thereof, so long as the mimic has a SNAIP activity as defined herein. That SNAIP activity can be any of those known, for example, binding a particular Wnt molecule, inducing a particular response in a cell, such as inhibiting apoptosis, and the like.

Thus, an agent that modulates SNAIP protein activity can be an agent as described herein, such as a nucleic acid or a protein, a naturally-occurring cognate ligand of a SNAIP protein, a peptide, a SNAIP peptidomimetic or other small molecule. In one embodiment, the agent stimulates one or more of the biological activities of SNAIP protein. Examples of such stimulatory agents include active SNAIP protein and a nucleic acid molecule encoding SNAIP that has been introduced into the cell. The modulatory methods can be performed in vitro (e.g., by culturing the cell with the agent) or, alternatively, in vivo (e.g., by administering the agent to a subject). As such, the instant invention provides methods of treating an individual afflicted with a disease or disorder characterized by aberrant or reduced expression or activity of a SNAIP protein or nucleic acid molecule. In one embodiment, the method involves administering an agent (e.g., an agent identified by a screening assay described herein) or combination of agents that modulates (e.g., upregulates or downregulates) SNAIP expression or activity. In another embodiment, the method involves administering a SNAIP protein or nucleic acid molecule as therapy to compensate for reduced or aberrant SNAIP expression or activity.

Stimulation of SNAIP activity is desirable in situations in which SNAIP is abnormally downregulated and/or in which SNAIP activity is decreased. Conversely, inhibition of SNAIP activity is decreased.

The invention is illustrated further by the following examples that should not be construed as limiting. The contents of all references, patents and published patent applications cited throughout the instant application hereby are incorporated by reference.

### EXAMPLES

RNA extraction: Cultured cells or tissues were lysed in 1.5 ml Trizol (Gibco, Cat. No. 15596) per 10 cm plate or 50 mg homogenized tissue, respectively. The lysate was passed through a pipette several times to homogenize the lysate (cell lysate subsequently was transferred to a tube). Following homogenization, the lysate was incubated for 5 minutes at 30° C. to permit the complete dissociation of nucleoprotein complexes. Following incubation, 0.2 ml of chloroform (Sigma, Catalog No. C53 12) per 1 ml of Trizol Reagent were added to the lysate and the tube was shakened vigorously for 15 seconds. The lysate then was incubated at 30° C. for 3 minutes. Following incubation, the lysate was centrifuged at 12,000×g for 15 minutes at 4° C. Following centrifugation, the supernatant was removed and the remaining RNA pellet was rinsed with 70% ethanol. The rinsed sample then was centrifuged at 7500×g for 10 minutes at 4° C. and the resulting supernatant was discarded. The remaining RNA pellet then was dried and resuspended in RNAase-free water (Life Technologies, Catalog No. 10977-015).

DNAse treatment: Total RNA was treated with DNAse I (Gibco) according to the manufacturer's protocol.

Differential display: First strand cDNA was synthesized from DNAse-treated total RNA using Advantage RT-for-PCR kit from Clontech. Two ug of total RNA were used per reaction. The cDNA product was diluted 1:10 and 1:100 and 1 μl of each dilution was used for the PCR reaction with arbitrary primers. Arbitrary primers from Hieroglyph and Fluoro DD primer kits (Beckman) were used. The primers contain oligodT or arbitrary sequences fused with either M13 or T7 parts. One set of primers was labeled with a fluorescent reporter. The PCR reactions were run using Advantage cDNA PCR kit (Clontech) according to the protocol recommended by the manufacturer. The fluorescent PCR products were separated on HR-1000 acrylamide gels (Beckman) using a GenomyxLR DNA Sequencer (Beckman). Samples from different experimental variants were run at least in duplicate and compared to the samples from controls. Gels were run at 1600 V for 6 hr, dried on the glass plate, washed several times to remove urea crystals and scanned using GenomyxSC scanner. Images were analyzed using Adobe Photoshop and coordinates of differentially expressed bands were determined. Using the coordinates, differentially expressed bands were located on dried gels. Bands were excised, soaked in 100 μl of water and spun down. Five μl of supernatant were used to reamplify the band in an PCR reaction using the Advantage polymerase mix and T7/M13 primers (Clontech). Reamplified bands either were sequenced directly using T7/M13 primers or cloned into the pCR2.1-TOPO vector from Invitrogen and then sequenced.

Reverse transcription: The reaction was performed using the RT for PCT kit from Clontech (Cat. No. K1402-1). One ug of RNA was isolated and DNased as described above, and then was mixed with 20 pmol of oligodT primer in a total volume of 13.5 μl. The mixture was incubated at 70° C. for 2 min and cooled to 4° C. for primer annealing. Following annealing, the 6.5 μl of reaction mix, containing reaction buffer, dNTP mix, RNAse inhibitor and MMLV reverse transcriptase from the RT for PCR kit were added and the PCR reaction conducted in a Perkin Elmer GeneAmp PCR System 9700 as described in manufacturer's protocols. The resulting cDNA product was stored at 20° C. until needed.

Real time PCR: TaqMan® or real time RT-PCR is a powerful tool for detecting messenger RNA in samples. The technology exploits the 5′ nuclease activity of AmpliTaq Gold® DNA polymerase to cleave a TaqMan® probe during PCT. The TaqMan® probe contains a reporter dye (in the experiments: 6-FAM (6-carboxyfluorescein)) at the 5′-end of the probe and a quencher dye (in the experiments: TAMRA (6-carboxy-N, N, N′, N′-tetramethylrhodamine)) at the 3′-end of the probe. TaqMan® probes are specifically designed to hybridize with the target cDNA of interest between the forward and the reverse primer sites. When the probe is intact, the 3′-end quencher dye suppresses the fluorescence of the 5′-end reporter dye. During PCR, the 5′→3′activity of the AmpliTaq Gold® DNA polymerase results in the cleavage of the probe between the 5′-end reporter dye and the 3′-end quencher dye resulting in the displacement of the reporter dye. Once displaced, the fluorescence of the reporter dye no longer is suppressed by the quencher dye. Thus, the accumulation of PCT products made from the targeted cDNA template is detected by monitoring the increase in fluorescence of the reporter dye.

An ABI Prism Sequence detector system from Perkin Elmer Applied Biosystems (Model No. ABI7700) was used to monitor the increase of the reporter fluorescence during PCR. The reporter signal is normalized to the emission of a passive reference. The RT-PCR reaction obtained as described above and diluted 1:100 with water was used as template in the TaqMan® assay.

Primers were designed using the Primer Express software (Perkin Elmer) and synthesized by Sigma Genosys. PCR reactions with each primer pair were run on a 4% agarose gel to confirm presence of a single band. The optimum final primer concentration in the reactions was found to be 0.2 μm for most primer pairs.

The TaqMan® assay was performed in a 96-well plate MicroAmp optical plate (Perkin Elmer, Catalog No. N801-0560). A reaction mixture comprising 25 μl of TaqMan® CybrGreen PCR Mixture (Perkin Elmer, Catalog No. 4309155), 2 μl forward primer, 2 μl of reverse primer, 5 μl CDNA and 17 μl of water were placed into each well. The plate then is sealed with MicroAmp optical 8-strip caps (Perkin Elmer, Catalog No. N801-0935). A separate Taqman reaction using primers for an arbitrary standard gene (e.g. beta actin, Perkin Elmer Cat. No. N801-0935) was performed for each experimental sample to permit normalization of results. Real time PCR reactions were run on the ABI Prizm System 7700 sequence detector (Perkin-Elmer).

RNA Labeling and Affymetrix Chip Hybridization: RNA labeling and chip hybridization were performed using standard Affymetrix procedures.

Analysis of Microarray Data: Analysis of microarray data was performed using Gecko (Aventis) and GeneSpring (Silicon Genetics) chip analysis software.

Neuroprotection Assay using human SNAIP: Human neuroblastoma cell lines SK-N-SH and SY5Y were seeded into 96 well plates and allowed to adhere overnight. Agents were tested in triplicates. Crude supernatants from 293 T cells transiently transfected with 1) full length SNAIP cDNA in Eukaryotic TopoTA plasmid without heparin, and 2) the same as (1) with heparin in the medium, 3) the empty vector control, 4) no vector control with and without heparin and 5) medium without 293 conditioning with and without heparin, were collected at 24 hrs and used at a 1:5 dilution. Positive controls for neuroprotection included flavopiridol used at 5 μm. Ten mM SIN-1 and 500 μm C2 ceramide were the neurotoxic agents added immediately after the neuroprotective agents. The plates were incubated overnight. Supernatants were collected and cell death determined using a lactate dehydrogenase (LDH) kit. SNAIP protected against SIN-1 and but not C2 ceramide neurotoxicity.

Cloning SNAIP: The gene sequence was amplified from pooled cortex and ventricular zone cDNAs using gene-specific primers synthesized by Sigma Genosys and the Advantage cDNA PCR kit (Clontech). The cDNA was cloned into an eukaryotic expression vector as known in the art. The cDNA was cloned in frame with the V5 epitope to allow detection of protein expression using commercially available V5 antibody (Invitrogen). The clone was also sequenced to confirm gene identity and absence of mutations.

Generation of Cells Expressing SNAIP: To provide significant quantities of SNAIP for further experiments, the cDNA encoding SNAIP was cloned into an expression vector and transfected into CHO cells.

To generate CHO cells overexpressing SNAIP, CHO cells were plated in a six-well 35 mm tissue culture plate 3×10⁵ cells per (Costar Catalog no. 3516) in 2 ml of F12 HAM media (Gibco/BRL, Catalog no. 11765-054) in the presence of 10% fetal bovine serum (Gibco/BRL Catalog No. 1600-044).

The cells then were incubated at 37° C. in a CO₂ incubator until the cells were 50-80% confluent. The cloned cDNA nucleic acid sequence of SNAIP was inserted using the procedure described above. Thirteen μg of the DNA were diluted into 1.2 ml of serum-free Optimem media with 78 μl PLUS reagent. Separately, 52 μl of Lipofectamine Plus Reagent (Life Technologies, Catalog No. 109064-013) was diluted into 1.25 ml of serum-free Optimem. The DNA solution and the Lipofectamine solution then were incubated at room temperature for 15 minutes. The two solutions were combined and incubated a further 15 minutes to allow for the formation of DNA-lipid complexes.

The cells were rinsed once with 2 ml of serum-free Optimem. For each transfection (six transfections in a six-well plate), medium on the cells was replaced with 0.8 ml Optimem. The DNA-lipid complex (hereinafter the “transfection mixture”) was added in a volume of 200 μl to each well. No anti-bacterial reagents were added. The cells then were incubated with the lipid-DNA complexes for 6 hours at 37° C. in a CO₂ incubator to allow for transfection.

After the completion of the incubation period, 1 ml of Optimem containing 20% fetal bovine serum was added onto the cells without first removing the transfection mixture. At 18 hours after transfection, the media overlaying the cells was aspirated. Cells then were washed with PBS pH 2-4 (Gibco/BRL Catalog No. 10010-023) and PBS was replaced with F12 HAM media containing 10% serum (“selective media”). At 72 hours after transfection, the cells were trypsinized and transferred to T150 flasks. Twenty four hours later medium was replaced with Ham's F12 with 10% FBS, antibiotics and 1 mg/ml G418. Selection continued for three days, then medium was replaced with medium containing 200 μg/ml G418.

Western blot analysis: Cell culture supernatant from transfected cell lines or lysed transfected cells were mixed with Invitrogen protein loading buffer and loaded on a 10% Tris-glycine gel from Invitrogen. The electrophoresis was run for 2.5 h at 100 V. After separation, the proteins were transferred to a PVDF membrane from Invitrogen for 1 h at 80V using the Invitrogen transfer apparatus. The membranes were blocked and hybridized with the anti-V5 antibody as described by the manufacturer (Invitrogen). A chemiluminescent substrate ECL (Cat. No. 1059250) and Hyperfilm ECL (Cat. No. HP79NA) from Amersham were used to detect protein band as described by Amersham.

Bands were visualized.

Although the instant invention has been described in detail with reference to the examples above, it is understood that various modifications can be made without departing from the spirit of the invention. Accordingly, the invention is limited only by the following claims.

All cited patents and publications referred to in this application are herein incorporated by reference in their entirety.

We claim:

1. A method of modulating peroxynitrite induced apoptosis in neuronal cells comprising contacting said cells with secreted neural apoptosis inhibiting protein (SNAIP).

2. The method of claim 1, wherein said method further comprises contacting said cells with heparin.

3. The method of claim 1, wherein said apoptosis comprises induction of the SIN-1 (3-morpholinosydonimine) peroxynitrite associated pathway.

4. The method of claim 3, wherein said pathway comprises activation of one or more of p38 MAPK, and growth arrest and DNA damage-inducible genes (GADDs).

5. The method of claim 3, wherein said pathway is detected by identifying a specific marker of protein nitration.

6. The method of claim 5, wherein said specific marker is 3 nitrotyrosine (3-NT).

7. The method of claim 4, wherein said GADDs are GADD34, GADD45 or GADD153.

8. The method of claim 1, wherein said apoptosis comprises mitochondrial dysfunction.

9. The method of claim 8, wherein said mitochondrial dysfunction comprises nitration of mitochondrial complex I subunits.

10. A method of protecting neurons from peroxynitrite-associated free radical-mediated cell death comprising contacting said cells with secreted neural apoptosis inhibiting protein.

11. A method of determining neuroprotective genomic targets associated with the peroxynitrite toxicity pathway, comprising:
i) contacting individual samples of neuronal cells each with and without secreted neural cell apoptosis inhibiting protein (SNAIP);
ii) contacting cells from step (i) with a peroxynitrite inducer;
iii) determining changes in expression of genes or proteins in cells of step (ii) and
iv) identifying genes or proteins modulated in the presence or absence of SNAIP and the inducer,
wherein genes or proteins so identified are correlated with inhibition of apoptosis induced by peroxynitrite induction.

12. The method of claim 11, wherein step (i) further comprises contacting said cells with or without heparin.

13. The method of claim 11, wherein said peroxynitrite inducer is SIN-1.

14. The method of claim 11, wherein said identified genes or proteins correlate with apoptosis.

15. A method for treating neuronal diseases associated with free radical mediated-cell death comprising administering to a patient in need thereof, a therapeutically effective amount of secreted neural apoptosis inhibiting protein (SNAIP).

16. The method of claim 15, wherein said diseases associated with said free radical-mediated cell death are selected from the group consisting of Parkinson's disease, multiple sclerosis, spinal cord injury, traumatic brain injury, stroke and Alzheimer's disease.

17. The method of claim 15, wherein said administration further comprises administration of heparin.
